(19) United States
(12) Patent Application Publication
(10) Pub. No.: US 2026/0216378 A1
(43) Pub. Date: Jul. 30, 2026

(54) NOVEL GENE THERAPY CONSTRUCT FOR CLN2 DISEASE

(71) Applicant: THE CHILDREN'S HOSPITAL OF PHILADELPHIA, Philadelphia, PA (US)

(72) Inventors: Beverly DAVIDSON, Philadelphia, PA (US); Paul RANUM, Philadelphia, PA (US); Yonghong CHEN, Philadelphia, PA (US); Megan S. KEISER, Philadelphia, PA (US); Luis TECEDOR, Philadelphia, PA (US)

(73) Assignee: THE CHILDREN'S HOSPITAL OF PHILADELPHIA, Philadelphia, PA (US)

(21) Appl. No.: 19/148,797

(22) PCT Filed: Jan. 30, 2024
(86) PCT No.: PCT/US2024/013505

§ 371 Date: Jul. 17, 2025

Related U.S. Application Data
(60) Provisional application No. 63/482,495, filed on Jan. 31, 2023.

(51) Int. Cl. A61K 48/00; A61K 9/00; A61P 25/28; C07K 14/005; C12N 9/48; C12N 15/86

(52) U.S. Cl. CPC A61K 48/0058; A61K 9/0085; A61K 48/0075; A61K 48/0083; A61P 25/28; C07K 14/005; C12N 9/485; C12N 15/86; C07K 2319/01; C12N 2750/14122; C12N 2750/14143; C12N 2830/50; C12Y 304/14009

(57) ABSTRACT

Provided herein are methods of expressing a tripeptidyl peptidase 1 (TPP1) in a neuron. Also provided herein is a modified adeno-associated virus (AAV) encoding a TPP1 under the control of a promoter operable in a neuron or ependyma cell, and not in dorsal root ganglia.

### PRIORITY CLAIM

This application claims benefit of priority to U.S. Provisional Application Ser. No. 63/482,495, filed Jan. 30, 2022, the entire contents of which are hereby incorporated by reference.

### BACKGROUND

### 1. Field

The present disclosure relates generally to the fields of medicine, virology, and neurology. More particularly, it concerns targeting peptides that target delivery of viral vectors to distinct structures in the brain, particularly in the treatment of TPP1 Deficiency/CLN2 disease.

### 2. Description of Related Art

TPP1 Deficiency/CLN2 disease is a childhood neurodegenerative disease caused by deficiency in the soluble lysosomal enzyme TPP1 due to mutations in CLN2. As patients with CLN2 cannot make any functional TPP1 on their own, gene replacement is the best therapeutic option. TPP1 is a mannose-6-phosphate decorated enzyme that can be used for cross-correction of deficient cells by enzyme replacement or gene therapy. For efficacy, gene therapy for TPP1 deficiency requires broad distribution throughout the brain. Transduction of ependyma cells which line the ventricles in CLN2-diseased dogs showed efficacy of treatment with improved neuropathology, prolonged lifespan, and amelioration of symptoms (Katz, et al, 2013). However, transduction of ependyma and deep brain structures in NHP brain is poorly achievable with current AAV serotypes.

Adeno-associated viruses (AAVs) represent strong therapeutic candidates for the treatment of neurodegenerative disease. AAVs are non-enveloped, single-stranded DNA viruses that can infect both dividing and non-dividing cells. Following infection, the virus does not exhibit robust integration within the host genome but persists as an episome in the cell nucleus. Expression of AAV cargoes is controlled spatially at the level of the packaging capsid and by the transgene promoter. Because use of AAV for the treatment of disease may necessitate intervention in diseased tissue, a problem can arise in that target tissue that contains a different gene expression profile than its healthy counterpart. Finding the correct promoter sequence to drive therapeutic transgene expression is an important goal.

Different strategies have been developed to generate AAV vector variants including rational design and directed evolution. The rational design approach utilizes knowledge of AAV capsids to make targeted changes to the capsid to alter transduction efficiency or specificity, such as tyrosine mutations on the capsid surface for increasing transduction efficiency. The directed evolution approach does not require any knowledge of capsid structure and is done through random mutagenesis, capsid shuffling, or random peptide insertions. These strategies generally use in vitro systems or mice, which are ideal for cell-based or mouse studies, but do not imply translation to the clinic. In fact, no AAV variants target distinct brain structures specifically or efficiently. As such, AAV variants that are capable to target distinct brain structures are needed.

### SUMMARY

Thus, in accordance with the present disclosure, there is provided a method of expressing a tripeptidyl peptidase 1 (TPP1) in a neuron or ependyma cell of a subject, comprising administering to the subject a modified adeno-associated virus (AAV) encoding a TPP1 under the control of a promoter operable in said neuron or ependymal cell, wherein said AAV comprises a modified capsid protein comprising a targeting sequence for targeting ependyma and CNS neurons, and wherein said modified AAV avoids transduction, of dorsal root ganglia. The promoter may be a constitutive promoter, or tissue specific promoter. The promoter may be a CMV early enhancer/chicken β actin (CAG) promoter.

The CAG promoter may comprise the sequence of SEQ ID NO: 1. The modified AAV may be AAV1. The targeting sequence may be inserted near or after position 590 of SEQ ID NO: 2. The targeting sequence may comprise or consist of ERDRTRG (SEQ ID NO: 3).

The TPP1 may comprise or consist of the sequence of SEQ ID NO: 4.

The targeting sequence may be flanked by linker sequences, wherein the linker sequences on each side of the targeting sequence are two or three amino acids long. The linker sequences may be SSA on the N-terminal side of the targeting sequence and AS on the C-terminal side of the targeting sequence. The modified capsid protein may comprise or consist of the sequence of SEQ ID NO: 5. The TPP1 sequence may be operably linked to a poly-adenylation signal.

Administering may be by direct intracerebroventricular or intracisternal magna or intrathecal delivery. The modified AAV may be administered more than once, such as 2, 3, 4, 5, 6, 7, 8, 9, 10 or more administrations. The modified AAV administration may occur monthly, annually, or every 5 years or with lower frequency.

A plurality of viral particles may be administered, such as by dosing at about 1×10⁶ to about 1×10¹⁴ vector genomes per kilogram (vg/kg), or dosing from about 1×10⁷-1×10¹⁴, about 1×10⁸-1×10¹⁴, about 1×10⁹-1×10¹⁴, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², or about 1×10¹²-1×10¹³ vg/kg of the patient.

The subject may suffer from late infantile neuronal ceroid lipofusincosis type 2 (CLN2) disease/Batten disease. The subject may be less than 4 years old or is between birth and 25 years old.

In another embodiment, there is provided a modified adeno-associated virus (AAV) encoding encoding a TPP1 under the control of a promoter operable in a neuron or ependyma cell, wherein said AAV comprises a modified capsid protein comprising a targeting sequence, and wherein said modified AAV does not target dorsal root ganglia. The promoter may be a constitutive promoter, or tissue specific promoter. The promoter may be a CMV early enhancer/chicken β actin (CAG) promoter.

The CAG promoter may comprise the sequence of SEQ ID NO: 1. The modified AAV may be based on AAV1 but with a targeting sequence near or after position 590 of SEQ ID NO: 2. The targeting sequence may comprise or consist of ERDRTRG (SEQ ID NO: 3).

The TPP1 may comprise or consist of the sequence of SEQ ID NO: 4.

The targeting sequence may be flanked by linker sequences, wherein the linker sequences on each side of the targeting sequence are two or three or more amino acids long. The linker sequences may be SSA on the N-terminal side of the targeting sequence and AS on the C-terminal side of the sequence peptide. The modified capsid protein may comprise or consist of the sequence of SEQ ID NO: 5. The TPP1 sequence may be operably linked to a poly-adenylation signal.

Also provided is a pharmaceutical composition comprising the modified AAV of as described herein as well as a kit comprising the modified AAV as described herein.

In yet another embodiment, there is provided a use of a modified adeno-associated virus (AAV) encoding a tripeptidyl peptidase 1 (TPP1) under the control of a promoter operable in a neuron or ependyma cell, wherein said AAV comprises a modified capsid protein comprising a targeting sequence such as ERDRTRG (SEQ ID NO: 3), for expressing TPP1 in a neuron or ependymal cell, wherein said modified AAV does not target dorsal root ganglia; a use of a modified adeno-associated virus (AAV) encoding a tripeptidyl peptidase 1 (TPP1) under the control of a promoter operable in a neuron or ependyma cell, wherein said AAV comprises a modified capsid protein comprising the targeting sequence ERDRTRG (SEQ ID NO: 3), for treating late infantile neuronal ceroid lipofusincosis type 2 (CLN2) disease/Batten disease in a subject afflicted therewith, wherein said modified AAV does not target dorsal root ganglia; and a use of a modified adeno-associated virus (AAV) encoding a tripeptidyl peptidase 1 (TPP1) under the control of a promoter operable in a neuron or ependyma cell, wherein said AAV comprises a modified capsid protein comprising the targeting sequence ERDRTRG (SEQ ID NO: 3), in the preparation of a medicament for treating late infantile neuronal ceroid lipofusincosis type 2 (CLN2) disease/Batten disease in a subject afflicted therewith, wherein said modified AAV does not target dorsal root ganglia.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. epAAV: Brain Parenchyma and ependyma shows strong signal throughout. An ependymal/neuron-targeting AAV capsid (epAAV) expressing the fluorophore mTFP1 (cyan) was injected introcerebroventricularly (ICV) to a non-human primate at 1.85E13 vg. Ependymal cells lining the lateral and fourth ventricles, including the choroid plexus had strong mTFP1 positive signal seen by confocal microscopy. In the brain parenchyma, there were positive cells in the subiculum and parasubiculum as well as in cortical areas throughout the brain (entorhinal, motor and temporal cortices). Positive mTFP1 signal was also seen in multiple nuclei of the thalamus (lateral dorsal, medial dorsal, and ventral anterior lateral thalamus). Confocal images were captured with a Leica SP8 microscope. Scale bar 50 μm.

FIG. 2. epAAV: Dorsal root ganglia (DRGs) are negative for our transgene. The ependymal-targeting AAV capsid (epAAV) expressing fluorophore mTFP1 (cyan) was injected introcerebroventricularly (ICV) to a non-human primate at 1.85E13 vg. Dorsal root ganglia toxicity is known to occur in the presence of strong exogenous expression from AAV delivery. Cell nuclei were visualized using counterstain with DAPI (blue; left column). RNAscope fluorescence in situ hybridization probed for transcripts of interest (middle column, probe denoted within image). A positive control (+control) was used to detect Ubiquitin C (UBC), a transcript known to be expressed in DRGs; a scrambled probe (Scramble) was used as a negative control (−control); and mTFP1 was detected with a probe to our transgene (mTFP1) in cervical, thoracic, and lumbar DRGs. Merged images are shown in the right column. There were no detectable transcripts in either the cervical, thoracic, or lumbar regions of DRGs. Thus, the ependyma/neuron targeting capsid does not transduce DRGs. Confocal images with a Leica SP8 microscope. Scale bar 50 μm.

FIG. 3. epAAV: Spinal Cord is negative for our transgene. An ependymal/neuronal-targeting AAV capsid (epAAV) expressing fluorophore mTFP1 (cyan) was injected introcerebroventricularly (ICV) to a non-human primate at 1.85E13 vg. Cell nuclei were visualized using counterstain with DAPI (blue; left column). RNAscope fluorescence in situ hybridization probed for transcripts of interest (middle column, probe denoted within image). A positive control (+control) was used to detect Ubiquitin C (UBC), a transcript known to be expressed in spinal cord; mTFP1 was detected with a probe to our transgene (mTFP1; cyan) in cervical, thoracic, and lumbar regions of the spinal cord. Merged images are shown in the right column. There were no detectable transcripts in either the cervical, thoracic, or lumbar regions of the spinal cord. Confocal images with a Leica SP8 microscope. Scale bar 50 μm.

FIG. 4. epAAV: Peripheral Organs show sparse to no positive cells. An ependymal/neuronal-targeting AAV capsid (epAAV) expressing fluorophore mTFP1 (cyan) was injected introcerebroventricularly (ICV) into the CSF to a non-human primate at 1.85E13 vg. Cell nuclei were visualized using counterstain with DAPI (blue; left column). Epifluorescence for mTFP1 was detected (cyan). Cells with high levels of autofluorescence accumulation are shown in red (autofluoresence). Merged images are shown in the right column. Liver (upper panels) had a low number of cells with positive signal for our transgene, mTFP1, whereas there was no detection of our transgene in the Heart (lower panels). All images were captured on a Leica DM6000B microscope. Scale bar 50 μm.

FIG. 5. Survival curve depicting an increase in life span in Cln2′/mice treated with our novel therapeutic construct relative to untreated normal or disease mice. Graph shows the percent survival of Cln2^{+/+}, Cln2^{-/-} and Cln2^{-/-}+5.0 E+10 vg epAAV mice over time. In general, the lowest survival rate was seen in Cln2^{-/-} mice (n=10) with an average of 17.1 weeks and lifespan maximum of 19.7 weeks. Gene therapy treatment with 5.0E+10 vg epAAV significantly increases Cln2^{-/-} (n=10) half-life until 18.6 weeks on average and a maximum of 28.6 weeks. 100% survival rate was seen in Cln2^{+/-} mice (n=9). * p<0.05 by Mantel-Cox test.

FIG. 6. Resting tremor phenotype onset is delayed and less aggressive in Cln2^{-/-} treated with our novel therapeutic construct relative to untreated. Graph shows a resting tremor assessment in Cln2^{+/+}, Cln2^{-/-} and Cln2^{-/-}+5.0 E+10 vg epAAV mice. Resting tremor phenotype can be detected in untreated Cln2^{-/-} mice at 13 weeks of age with respect to Cln2^{+/+}, with aggressive increase in severity over time. Cln2^{-/-} mice treated with 5.0E+10vg epAAV significantly delayed onset of resting tremor until 16 weeks, with a notable attenuation of phenotype and slower progression over time.

FIG. 7. TPP1 activity in the central nervous system is increased relative to endogenous levels after treatment with novel therapeutic construct. Graphs show TPP1 activity in Cln2^{+/-} and Cln2^{-/-} mice untreated or treated with a unilateral injection of 5.0 E+10 vg epAAV. epAAV injected mice have increased TPP1 activity in the different analyzed brain areas when compared to endogenous TPP1 activity in uninjected Cln2^{-/-} mice and are also greatly elevated over normal mice. The heart and spleen of injected Cln2^{-/-} mice, though not the direct target of therapy, have minimally increased levels of TPP1 activity relative to uninjected Cln2^{-/-} mice.

FIGS. 8A-D. Quantification of ependymal transduction in 5 NHP that received ICV infusion of AAV-EP+. AAV-Ep+ administered to (n=5) rhesus macaques via intracerebral ventricular (ICV) infusion at 1E13 vg per subject. Transduced ependymal cells and parenchymal neurons identified by fluorescence and morphology via microscopy of sectioned brains. Total and transduced numbers of cells counted via an automated algorithm, using Cellpose and QuPath. (FIG. 8A) Representative 40× image showing robust transduction and mRuby3 expression of the ependymal layer of cells lining the ventricle. (FIG. 8B) Quantification of transduced ependymal and parenchymal cells across 75 images (40×), 15,096 total cells, and 5,852 ependymal cells. (FIG. 8C) 10× tilescan images taken showing broad ependymal transduction across the area of the anterior (slab 4) lateral ventricle ependymal lining. (FIG. 8D) Quantification of 10× tilescan images from lateral ventricle in slabs 4 and 5 of 4 NHPs. 8 tilescans, and 34,734 total ependymal cells were quantified.

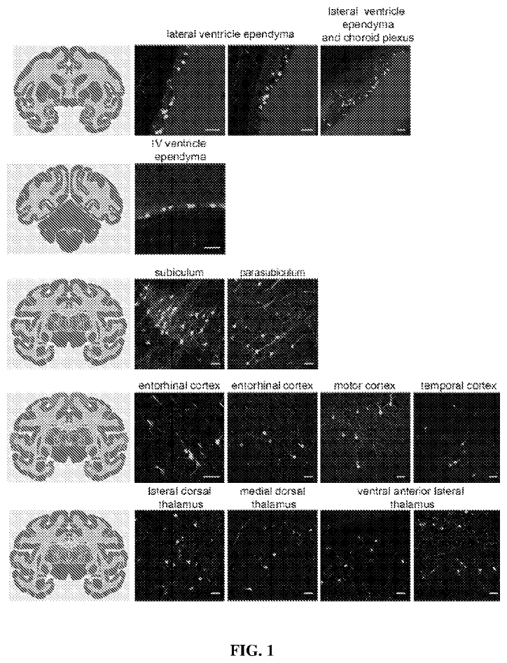

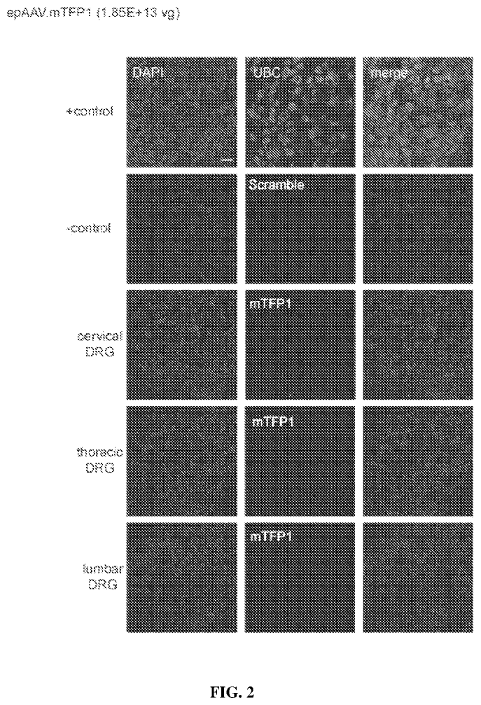

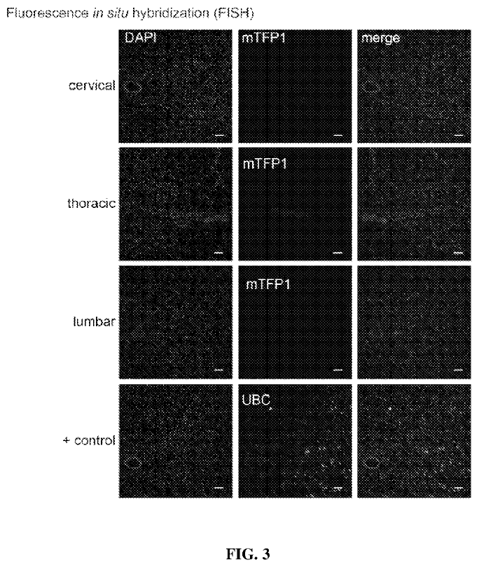

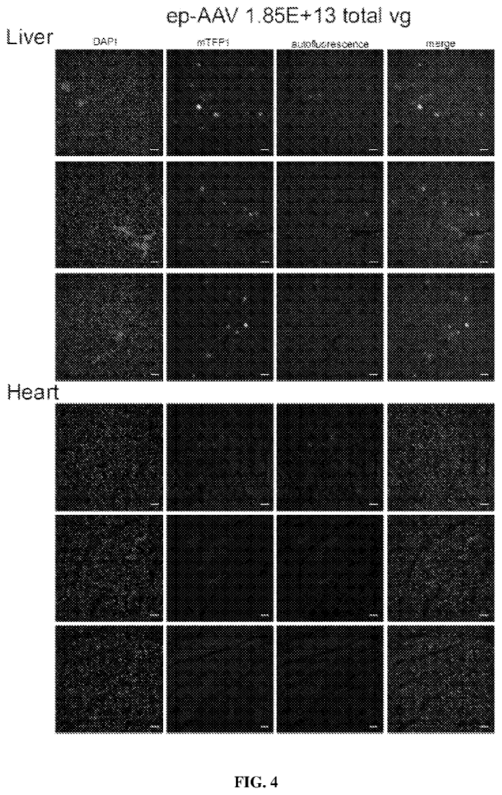

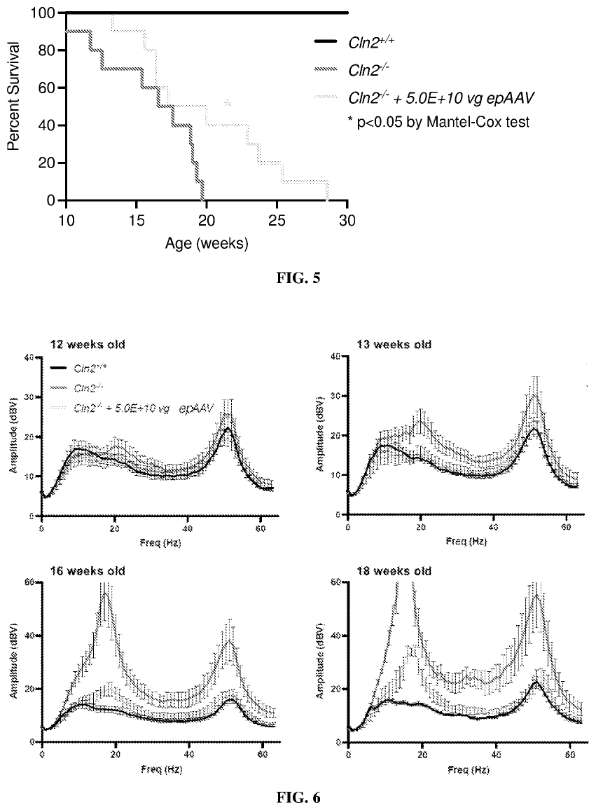

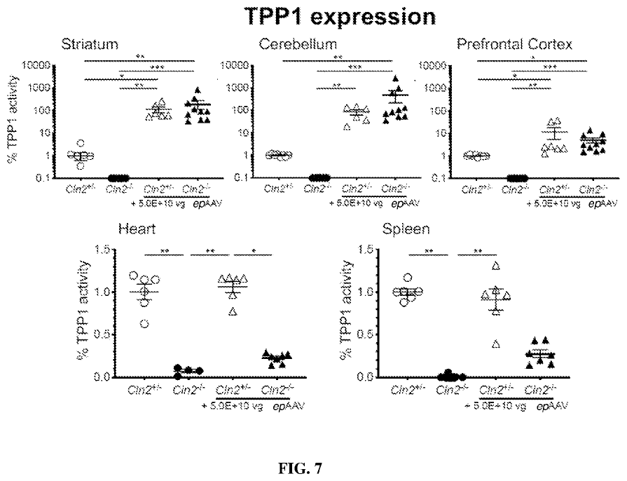

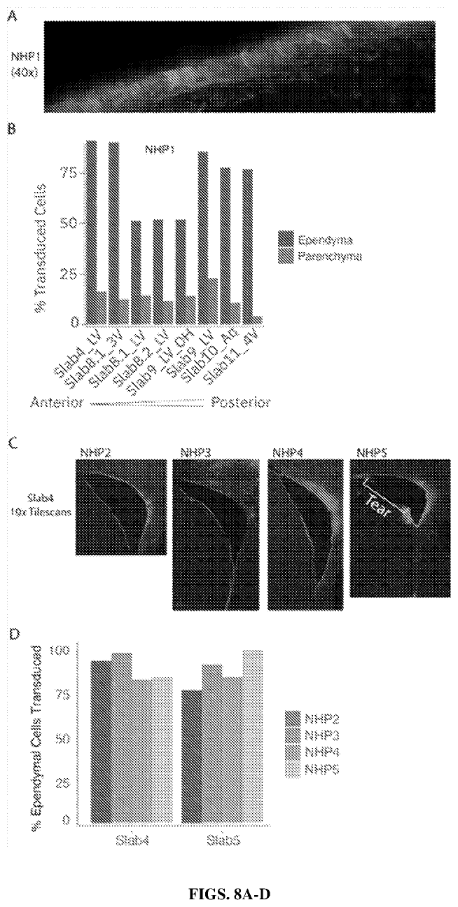

### DETAILED DESCRIPTION

Provided herein are methods of expressing a tripeptidyl peptidase 1 (TPP1) in a the ependyma and neurons of a subject, comprising administering to the subject a modified adeno-associated virus (AAV) capsid encapsidating a transgene encoding a TPP1 under the control of a promoter operable in said neuron/ependyma, wherein said AAV comprises a modified capsid protein comprising a targeting sequence.

In some embodiments, viral vectors each comprise a modified capsid, wherein the modified capsid comprises an amino acid sequence that targets the viral vector to a distinct brain structure. In certain embodiments, the brain structure is the brainstem, caudate, cerebellar cortex, cerebral cortex, ependyma, globus pallidus, hippocampus, meninges, optic nerve, putamen, spinal cord, substantia nigra, subthalamic nuclei, thalamus or ependymal cells. In certain embodiments, the targeting peptide is ERDRTRG (SEQ ID NO: 3).

In certain embodiments, the viral vector is an adeno associated viral vector (AAV). In certain embodiments, the AAV is derived from AAV1. An exemplary wildtype reference AAV1 capsid protein sequence is provided in SEQ ID NO: 1, which does not transduce ependyma to any appreciable degree after delivery into the CSF. In certain aspects, the targeting peptide is inserted at position 590 of the AAV1 capsid. An exemplary modified AAV1 capsid protein sequence is provided in SEQ ID NO: 4, which shows the targeting peptide insertion after position 590 as SSAX₇AS, where the leading SSA and the trailing AS are linker sequences and X₇ represents the targeting peptide.

### I. BATTEN DISEASE/TPPI DEFICIENCY

In some aspects, the present disclosure may be directed to the use of treating a neurological disorder, such as late infantile neuronal ceroid lipofusincosis type 2 (CLN2) disease/Batten disease. Batten disease is a fatal disease of the nervous system that typically begins in childhood, typically between 5 and 10 years of age. Often, it is autosomal recessive, a common name for the neuronal ceroid lipofuscinoses (NCLs). Although Batten disease is usually regarded as the juvenile form of NCL (or “type 3”), some physicians use the term Batten disease to describe all forms of NCL. Historically, the NCLs were classified by age of disease onset as infantile NCL (INCL), late infantile NCL (LINCL), juvenile NCL (JNCL) or adult NCL (ANCL). At least 20 genes have been identified in association with Batten disease, but juvenile NCL, the most prevalent form of Batten disease, has been linked to mutations in the CL 3 gene.

Batten disease is a terminal illness. Brineura is the first FDA-approved treatment to slow loss of walking ability (ambulation) in symptomatic pediatric patients 3 years of age and older with late infantile neuronal ceroid lipofuscinosis type 2 (CLN2), also known as tripeptidyl peptidase-1 (TPP1) deficiency. TPP1, also known as Lysosomal pepstatin-insensitive protease, is an enzyme that in humans is encoded by the TPP1 gene. Mutations in the TPP1 gene leads to late infantile neuronal ceroid lipofuscinosis. The human gene TPP1 encodes a member of the sedolisin family of serine proteases. The human gene has 13 exons and locates at the chromosome band 11p15.

The nucleic acid sequence of human TPP1 is set forth as SEQ ID NO: 4. Human TPP1 comprises tripeptidyl-peptidase I activity (TPP1 enzyme activity). TPP1 activity comprises a non-specific lysosomal peptidase activity which generates tripeptides from the breakdown products produced by lysosomal proteinases. Substrate-specificity studies indicate that TPP1 primarily cleaves tripeptides from unsubstituted amino termini in peptides and proteins. Endogenously expressed TPP1 is synthesized as a catalytically inactive enzyme. After targeting lysosomes, because of the acidic environment, TPP1 is auto-catalytically processed into a mature active enzyme. The activity of TPP1 can be measured and/or quantitated in vitro using known methods. See, for example, Junaid et al., 1999.

The human TPP1 is 61 kDa in size and composed of 563 amino acids. An isoform of 34.5 kDa and 320 amino acids is generated by alternative splicing and a peptide fragment of 1-243 amino acid is missing. TPP1 contains a globular structure with a subtilisin-like fold, a Ser 475-Glu 272-Asp 360 catalytic triad. It also contains an octahedrally coordinated Ca²⁺-binding site that are characteristic features of the S53 sedolisin family of peptidases. Unlike other S53 peptidases, it has steric constraints on the P4 substrate pocket, which might contribute to its preferential cleavage of tripeptides from the unsubstituted N-terninus of proteins. Two aternative conformations of the catalytic Asp276 are associated with the activation status of TPPL.

High expression of TPP1 is found in bone marrow, placenta, lung, pineal and lymphocytes. The protease functions in the lysosome to cleave N-terminal tripeptides from substrates and has weaker endopeptidase activity. It is synthesized as a catalytically inactive enzyme which is activated and autoproteolyzed upon acidification.

The neuronal ceroid lipofuscinoses (NCLs) are a group of inherited neurodegenerative disorders with pathological phenotypes that auto fluorescent lipopigments present in neurons and other cell types. Over the past two decades, accumulating evidence indicates that NCLs are caused by mutations in eight different genes, including genes encoding several soluble proteins (cathepsin D, PPT1 and TPP1). Mutations of gene TPP1 result in late-infantile neuronal ceroid lipofuscinosis which is associated with the failure to degrade specific neuropeptides and a subunit of ATP synthase in the lysosome. Mutations in the TPP1 gene lead to late infantile neuronal ceroid lipofuscinosis, a fatal neurodegenerative disease of childhood. It has been demonstrated that a single injection of intravitreal implantation of autologous bone marrow derived stem cells transduced with a TPP1 expression construct at an early stage in the disease progression could substantially inhibit the development of disease-related retinal function deficits and structural changes. This result implies that ex vivo gene therapy using autologous stem cells may be an effective means of achieving sustained delivery of therapeutic compounds to tissues such as the retina for which systemic administration would be ineffective.

In some embodiments, TPP1 may be under the control of a promoter, such as a CMV early enhancer/chicken β actin (CAG) promoter. The CAG promoter is a strong synthetic promoter frequently used to drive high levels of gene expression in mammalian expression vectors. The CAG promoter was constructed from the following sequences:
(C) the cytomegalovirus (CMV) early enhancer element,
(A) the promoter, the first exon and the first intron of chicken beta-actin gene,
(G) the splice acceptor of the rabbit beta-globin gene

The resulting synthetic element was used in the pCAGGS expression vector. The initiation codon located at the proximal region of the second exon was disrupted by digesting with NcoI restriction enzyme and replacing the site with a HindIII linker. Although the whole construct is commonly referred to as the “CAG promoter”, it includes a part of the transcribed sequence (the first exon and the first intron of chicken beta-actin gene) and enhancer elements. In addition to the CMV immediate early enhancer, the intron of the chicken beta actin gene contains an enhancer element, which is highly conserved among vertebrates. The 3′ portion of the promoter has high GC content and is thus refractory to PCR amplification.

### II. ADENO-ASSOCIATED VIRUS (AAV) VECTORS

Adeno-associated virus (AAV) is a small nonpathogenic virus of the parvoviridae family. To date, numerous serologically distinct AAVs have been identified, and more than a dozen have been isolated from humans or primates. AAV is distinct from other members of this family by its dependence upon a helper virus for replication.

AAV genomes can exist in an extrachromosomal state without integrating into host cellular genomes; possess a broad host range; transduce both dividing and non-dividing cells in vitro and in vivo and maintain high levels of expression of the transduced genes. AAV viral particles are heat stable; resistant to solvents, detergents, changes in pH, and temperature; and can be column purified and/or concentrated on CsCl gradients or by other means. The AAV genome comprises a single-stranded deoxyribonucleic acid (ssDNA), either positive- or negative-sensed. The approximately 4.7 kb genome of AAV consists of one segment of single stranded DNA of either plus or minus polarity. The ends of the genome are short-inverted terminal repeats (ITRs) that can fold into hairpin structures and serve as the origin of viral DNA replication.

An AAV “genome” refers to a recombinant nucleic acid sequence that is ultimately packaged or encapsulated to form an AAV particle. An AAV particle often comprises an AAV genome packaged with AAV capsid proteins. In cases where recombinant plasmids are used to construct or manufacture recombinant vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for plasmid propagation and production but is not itself packaged or encapsulated into viral particles. Thus, an AAV vector “genome” refers to nucleic acid that is packaged or encapsulated by AAV capsid proteins.

The AAV virion (particle) is a non-enveloped, icosahedral particle approximately 25 nm in diameter that comprises an AAV capsid. The AAV particle comprises an icosahedral symmetry comprised of three related capsid proteins, VP1, VP2 and VP3, which interact together to form the capsid. The genome of most native AAVs often contain two open reading frames (ORFs), sometimes referred to as a left ORF and a right ORE. The right ORF often encodes the capsid proteins VP1, VP2, and VP3. These proteins are often found in a ratio of 1:1:10 respectively, but may be in varied ratios, and are all derived from the right-hand ORE. The VP1, VP2 and VP3 capsid proteins differ from each other by the use of alternative splicing and an unusual start codon. Deletion analysis has shown that removal or alteration of VP1 which is translated from an alternatively spliced message results in a reduced yield of infectious particles. Mutations within the VP3 coding region result in the failure to produce any single-stranded progeny DNA or infectious particles. In certain embodiments, the genome of an AAV particle encodes one, two or all three VP1, VP2 and VP3 polypeptides.

The left ORF often encodes the non-structural Rep proteins, Rep 40, Rep 52, Rep 68 and Rep 78, which are involved in regulation of replication and transcription in addition to the production of single-stranded progeny genomes. Two of the Rep proteins have been associated with the preferential integration of AAV genomes into a region of the q arm of human chromosome 19. Rep68/78 have been shown to possess NTP binding activity as well as DNA and RNA helicase activities. Some Rep proteins possess a nuclear localization signal as well as several potential phosphorylation sites. In certain embodiments the genome of an AAV (e.g., an rAAV) encodes some or all of the Rep proteins. In certain embodiments the genome of an AAV (e.g., an rAAV) does not encode the Rep proteins. In certain embodiments one or more of the Rep proteins can be delivered in trans and are therefore not included in an AAV particle comprising a nucleic acid encoding a polypeptide.

The ends of the AAV genome comprise short-inverted terminal repeats (ITR) which have the potential to fold into T-shaped hairpin structures that serve as the origin of viral DNA replication. Accordingly, the genome of an AAV comprises one or more (e.g., a pair of) ITR sequences that flank a single stranded viral DNA genome. The ITR sequences often have a length of about 145 bases each. Within the ITR region, two elements have been described which are believed to be central to the function of the ITR, a GAGC repeat motif and the terminal resolution site (trs). The repeat motif has been shown to bind Rep when the ITR is in either a linear or hairpin conformation. This binding is thought to position Rep68/78 for cleavage at the trs which occurs in a site- and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The term “recombinant,” as a modifier of vector, such as recombinant viral, e.g., lentivirus or parvovirus (e.g., AAV) vectors, as well as a modifier of sequences such as recombinant nucleic acid sequences and polypeptides, means that the compositions have been manipulated (i.e., engineered) in a fashion that generally does not occur in nature. A particular example of a recombinant vector, such as an AAV, retroviral, or lentiviral vector would be where a nucleic acid sequence that is not normally present in the wild-type viral genome is inserted within the viral genome. An example of a recombinant nucleic acid sequence would be where a nucleic acid (e.g., gene) encodes an inhibitory RNA cloned into a vector, with or without 5′, 3′ and/or intron regions that the gene is normally associated within the viral genome. Although the term “recombinant” is not always used herein in reference to vectors, such as viral vectors, as well as sequences such as polynucleotides, “recombinant” forms including nucleic acid sequences, polynucleotides, transgenes, etc. are expressly included in spite of any such omission.

A recombinant viral “vector” is derived from the wild-type genome of a virus by using molecular methods to remove part of the wild-type genome from the virus, and replacing it with a non-native nucleic acid, such as a nucleic acid sequence. Typically, for example, for AAV, one or both inverted terminal repeat (ITR) sequences of the AAV genome are retained in the recombinant AAV vector. A “recombinant” viral vector (e.g., rAAV) is distinguished from a viral (e.g., AAV) genome, since part of the viral genome has been replaced with a non-native sequence with respect to the viral genomic nucleic acid such a nucleic acid encoding a transactivator or nucleic acid encoding an inhibitory RNA or nucleic acid encoding a therapeutic protein. Incorporation of such non-native nucleic acid sequences therefore defines the viral vector as a “recombinant” vector, which in the case of AAV can be referred to as a “rAAV vector.”

In certain embodiments, an AAV (e.g., a rAAV) comprises two ITRs. In certain embodiments, an AAV (e.g., a rAAV) comprises a pair of ITRs. In certain embodiments, an AAV (e.g., a rAAV) comprises a pair of ITRs that flank (i.e., are at each 5′ and 3′ end) of a nucleic acid sequence that at least encodes a polypeptide having function or activity.

An AAV vector (e.g., rAAV vector) can be packaged and is referred to herein as an “AAV particle” for subsequent infection (transduction) of a cell, ex vivo, in vitro or in vivo. Where a recombinant AAV vector is encapsulated or packaged into an AAV particle, the particle can also be referred to as a “rAAV particle.” In certain embodiments, an AAV particle is a rAAV particle. A rAAV particle often comprises a rAAV vector, or a portion thereof. A rAAV particle can be one or more rAAV particles (e.g., a plurality of AAV particles). rAAV particles typically comprise proteins that encapsulate or package the rAAV vector genome (e.g., capsid proteins). It is noted that reference to a rAAV vector can also be used to reference a rAAV particle.

Any suitable AAV particle (e.g., rAAV particle) can be used for a method or use herein. A rAAV particle, and/or genome comprised therein, can be derived from any suitable serotype or strain of AAV. A rAAV particle, and/or genome comprised therein, can be derived from two or more serotypes or strains of AAV. Accordingly, a rAAV can comprise proteins and/or nucleic acids, or portions thereof, of any serotype or strain of AAV, wherein the AAV particle is suitable for infection and/or transduction of a mammalian cell. Non-limiting examples of AAV serotypes include AAV1, AAV2, AAV3, AAV4, AAV5, AAV6, AAV7, AAV8, AAV9, AAV10, AAV11, AAV12, AAV-rh74, AAV-rh10 and AAV-2i8.

In certain embodiments a plurality of rAAV particles comprises particles of, or derived from, the same strain or serotype (or subgroup or variant). In certain embodiments a plurality of rAAV particles comprise a mixture of two or more different rAAV particles (e.g., of different serotypes and/or strains).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

In certain embodiments, a rAAV vector based upon a first serotype genome corresponds to the serotype of one or more of the capsid proteins that package the vector. For example, the serotype of one or more AAV nucleic acids (e.g., ITRs) that comprises the AAV vector genome corresponds to the serotype of a capsid that comprises the rAAV particle.

In certain embodiments, a rAAV vector genome can be based upon an AAV (e.g., AAV2) serotype genome distinct from the serotype of one or more of the AAV capsid proteins that package the vector. For example, a rAAV vector genome can comprise AAV1 derived nucleic acids (e.g., ITRs), whereas at least one or more of the three capsid proteins are derived from a different serotype, e.g., an AAV1, AAV3, AAV4, AAV5, AAV6, AAV7, AAV8, AAV9, AAV10, AAV11, AAV12, Rh10, Rh74 or AAV-2i8 serotype or variant thereof.

In certain embodiments, a rAAV particle or a vector genome thereof related to a reference serotype has a polynucleotide, polypeptide or subsequence thereof that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a polynucleotide, polypeptide or subsequence of an AAV1, AAV2, AAV3, AAV4, AAV5, AAV6, AAV7, AAV8, AAV9, AAV10, AAV11, AAV12, Rh10, Rh74 or AAV-2i8 particle. In particular embodiments, a rAAV particle or a vector genome thereof related to a reference serotype has a capsid or ITR sequence that comprises or consists of a sequence at least 60% or more (e.g., 65%, 70%, 75%, 80%, 85%, 90%, 95%, 96%, 97%, 98%, 99%, 99.1%, 99.2%, 99.3%, 99.4%, 99.5%, etc.) identical to a capsid or ITR sequence of an AAV1, AAV2, AAV3, AAV4, AAV5, AAV6, AAV7, AAV8, AAV9, AAV10, AAV11, AAV12, Rh10, Rh74 or AAV-2i8 serotype.

In certain embodiments, a method herein comprises use, administration or delivery of an rAAV1, rAAV2, rAAV3, rAAV4, rAAV5, rAAV6, rAAV7, rAAV8, rAAV9, rAAV10, rAAV11, rAAV12, rRh10, rRh74 or rAAV-2i8 particle.

In certain embodiments, a method herein comprises use, administration or delivery of a rAAV1 particle. In certain embodiments a rAAV1 particle comprises an AAV1 capsid. In certain embodiments a rAAV1 particle comprises one or more capsid proteins (e.g., VP1, VP2 and/or VP3) that are at least 60%, 65%, 70%, 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV1 particle. In certain embodiments a rAAV1 particle comprises VP1, VP2 and VP3 capsid proteins that are at least 75% or more identical, e.g., 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to a corresponding capsid protein of a native or wild-type AAV1 particle. In certain embodiments, a rAAV2 particle is a variant of a native or wild-type AAV1 particle. In some aspects, one or more capsid proteins of an AAV2 variant have 1, 2, 3, 4, 5, 5-10, 10-15, 15-20 or more amino acid substitutions compared to capsid protein(s) of a native or wild-type AAV1 particle.

In certain embodiments, a rAAV particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1, AAV2, AAV3, AAV4, AAV5, AAV6, AAV7, AAV8, AAV9, AAV10, AAV11, AAV12, AAV-rh74, AAV-rh10 or AAV-2i8, as long as they retain one or more desired ITR functions (e.g., ability to form a hairpin, which allows DNA replication; integration of the AAV DNA into a host cell genome; and/or packaging, if desired).

In certain embodiments, a rAAV1 particle comprises one or two ITRs (e.g., a pair of ITRs) that are at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to corresponding ITRs of a native or wild-type AAV1 particle, as long as they retain one or more desired ITR functions (e.g., ability to form a hairpin, which allows DNA replication; integration of the AAV DNA into a host cell genome; and/or packaging, if desired).

A rAAV particle can comprise an ITR having any suitable number of “GAGC” repeats. In certain embodiments an ITR of an AAV2 particle comprises 1, 2, 3, 4, 5, 6, 7, 8, 9 or 10 or more “GAGC” repeats. In certain embodiments a rAAV2 particle comprises an ITR comprising three “GAGC” repeats. In certain embodiments a rAAV2 particle comprises an ITR which has less than four “GAGC” repeats. In certain embodiments a rAAV2 particle comprises an ITR which has more than four “GAGC” repeats. In certain embodiments an ITR of a rAAV2 particle comprises a Rep binding site wherein the fourth nucleotide in the first two “GAGC” repeats is a C rather than a T.

Exemplary suitable length of DNA can be incorporated in rAAV vectors for packaging/encapsidation into a rAAV particle can about 5 kilobases (kb) or less. In particular, embodiments, length of DNA is less than about 5 kb, less than about 4.5 kb, less than about 4 kb, less than about 3.5 kb, less than about 3 kb, or less than about 2.5 kb.

rAAV vectors that include a nucleic acid sequence that directs the expression of an RNAi or polypeptide can be generated using suitable recombinant techniques known in the art (e.g., see Sambrook et al., 1989). Recombinant AAV vectors are typically packaged into transduction competent AAV particles and propagated using an AAV viral packaging system. A transduction competent AAV particle is capable of binding to and entering a mammalian cell and subsequently delivering a nucleic acid cargo (e.g., a heterologous gene) to the nucleus of the cell. Thus, an intact rAAV particle that is transduction-competent is configured to transduce a mammalian cell. A rAAV particle configured to transduce a mammalian cell is often not replication competent and requires additional protein machinery to self-replicate. Thus, a rAAV particle that is configured to transduce a mammalian cell is engineered to bind and enter a mammalian cell and deliver a nucleic acid to the cell, wherein the nucleic acid for delivery is often positioned between a pair of AAV ITRs in the rAAV genome.

Suitable host cells for producing transduction competent AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments and expresses the adenoviral Ela and Elb genes is used to generate recombinant AAV particles. The modified HEK293 cell line is readily transfected and provides a particularly convenient platform in which to produce rAAV particles. Methods of generating high titer AAV particles capable of transducing mammalian cells are known in the art.

In certain embodiments, AAV helper functions are introduced into the host cell by transfecting the host cell with an AAV helper construct either prior to, or concurrently with, the transfection of an AAV expression vector. AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

### III. AAV-TTP1 AS A THERAPEUTIC AGENT

In some embodiments, viral gene transfer methods can be used to introduce nucleic acids in mammalian cells. Such methods can be used to administer nucleic acids encoding therapeutic proteins to cells in culture or in a host organism. In some embodiments, the therapeutic proteins may be useful for the treatment of late infantile neuronal ceroid lipofusincosis type 2 (CLN2) disease/Batten disease. Some embodiments may concern the expression of a polypeptide comprising tripeptidyl peptidase 1 (TPP1) activity.

A polypeptide comprising TPP1 activity refers to a TPP1 protein of a mammal, or a portion thereof, that displays at least 50%, at least 60%, at least 70%, at least 75%, at least 80%, at least 85%, at least 90%, at least 95%, or about 100% of the peptidase activity of the human TPP1 of SEQ ID NO: 4 as assayed using a suitable peptide substrate, for example, as assayed by the method of Junaid et al., 1999 or another comparable method. In certain embodiments, a polypeptide comprising TPP1 activity refers to a TPP1 protein of a mammal, or a subsequence or variant thereof, that displays at least at least 50%, at least 60%, at least 70%, at least 75%, at least 80%, at least 85%, at least 90%, at least 95%, or about 100% of the peptidase activity of the human TPP1 of SEQ ID NO: 4.

A polypeptide comprising TPP1 activity may comprise a truncated, mutated, chimeric, or modified form of a TPP1 polypeptide that retains at least partial TPP1 activity. A polypeptide comprising TPP1 activity may comprise a TPP1 protein, or a portion thereof, obtained from any suitable organism (e.g., from a mammal, from a human, from a non-human mammal, e.g., from a dog, pig, cow, or the like). In certain embodiments a polypeptide comprising TPP1 activity has at least 60% identity, at least 70% identity, at least 75% identity, at least 80% identity, at least 85% identity, at least 90% identity, at least 95% identity, at least 98% identity, or 100% identity to the TPP1 encoded by the sequence set forth in SEQ ID NO: 4.

In certain embodiments, an AAV particle comprises an AAV capsid protein and a nucleic acid encoding a polypeptide comprising TPP1 activity. In certain embodiments, an AAV particle comprises an AAV capsid protein and a nucleic acid that directs the expression and/or secretion of a polypeptide comprising TPP1 activity. In certain embodiments, an AAV particle comprises an AAV capsid protein and a nucleic acid encoding a TPP1 polypeptide, or enzymatically active portion thereof. In certain embodiments, an AAV particle comprises an AAV capsid protein and a nucleic acid that directs the expression and/or secretion of a TPP1 polypeptide, or enzymatically active portion thereof. In certain embodiments, an AAV particle comprises a polypeptide having at least 50% identity, at least 60% identity, at least 70% identity, at least 75% identity, at least 80% identity, at least 85% identity, at least 90% identity, at least 95% identity, at least 98% identity, or 100% identity to the sequence set forth in SEQ ID NO: 2 or SEQ ID NO: 5. In certain embodiments, a nucleic acid being administered encodes TPP1, a TPP1 that has substantial identity to wild-type TPP1, and/or a variant, mutant or fragment of a TPP1. In certain embodiments a nucleic acid encoding a TPP1 activity or encoding or directing the expression of a TPP1 polypeptide is a nucleic acid having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO: 4.

Recombinant TTP1 polypeptides may possess deletions and/or substitutions of amino acids; thus, a protein with a deletion, a protein with a substitution, and a protein with a deletion and a substitution are modified proteins. In some embodiments, these proteins may further include insertions or added amino acids, such as with fusion proteins or proteins with linkers, for example. A “modified deleted protein” lacks one or more residues of the native protein but may possess the specificity and/or activity of the native protein.

Substitution or replacement variants typically contain the exchange of one amino acid for another at one or more sites within the protein and may be designed to modulate one or more properties of the polypeptide, particularly its effector functions and/or bioavailability. Substitutions may or may not be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine, or methionine; serine to threonine; threonine to serine; tryptophan to tyrosine; tyrosine to tryptophan or phenylalanine; and valine to isoleucine or leucine.

In addition to a deletion or substitution, a modified protein may possess an insertion of residues, which typically involves the addition of at least one residue in the polypeptide. This may include the insertion of a targeting peptide or polypeptide or simply a single residue.

The term “biologically functional equivalent” is well understood in the art and is further defined in detail herein. Accordingly, sequences that have between about 70% and about 80%, or between about 81% and about 90%, or even between about 91% and about 99% of amino acids that are identical or functionally equivalent to the amino acids of a control polypeptide are included, provided the biological activity of the protein is maintained. A recombinant protein may be biologically functionally equivalent to its native counterpart in certain aspects.

It also will be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region or may include various internal sequences, i.e., introns, which are known to occur within genes.

### IV. METHODS OF ADMINISTRATION

Viral vectors, in some aspects, may be administered directly to patients (in vivo) or they can be used to treat cells in vitro or ex vivo, and then administered to patients. The term “vector” refers to small carrier nucleic acid molecule, a plasmid, virus (e.g., AAV vector), or other vehicle that can be manipulated by insertion or incorporation of a nucleic acid. Vectors, such as viral vectors, can be used to introduce/transfer nucleic acid sequences into cells, such that the nucleic acid sequence therein is transcribed and, if encoding a protein, subsequently translated by the cells.

Any suitable cell or mammal can be administered or treated by a method or use described herein. Typically, a mammal in need of a method described herein is suspected of having or expressing an abnormal or aberrant protein that is associated with a disease state. Alternatively, the mammalian recipient may have a condition that is amenable to gene replacement therapy. As used herein, “gene replacement therapy” refers to administration to the recipient of exogenous genetic material encoding a therapeutic agent and subsequent expression of the administered genetic material in situ. Thus, the phrase “condition amenable to gene replacement therapy” embraces conditions such as genetic diseases (i.e., a disease condition that is attributable to one or more gene defects), acquired pathologies (i.e., a pathological condition which is not attributable to an inborn defect), cancers and prophylactic processes (i.e., prevention of a disease or of an undesired medical condition). Accordingly, as used herein, the term “therapeutic agent” refers to any agent or material, which has a beneficial effect on the mammalian recipient. Thus, “therapeutic agent” embraces both therapeutic and prophylactic molecules having nucleic acid or protein components.

Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In certain embodiments a mammal is a human. In certain embodiments a mammal is a non-rodent mammal (e.g., human, pig, goat, sheep, horse, dog, or the like). In certain embodiments a non-rodent mammal is a human. A mammal can be any age or at any stage of development (e.g., an adult, teen, child, infant, or a mammal in utero). A mammal can be male or female. In certain embodiments a mammal can be an animal disease model, for example, animal models having or expressing an abnormal or aberrant protein that is associated with a disease state or animal models with insufficient expression of a protein, which causes a disease state.

Mammals (subjects) treated by a method or composition described herein include adults (18 years or older) and children (less than 18 years of age). Adults include the elderly. Representative adults are 50 years or older. Children range in age from 1-2 years old, or from 2-4, 4-6, 6-18, 8-10, 10-12, 12-15 and 15-18 years old. Children also include infants. Infants typically range from 1-12 months of age.

In certain embodiments, a method includes administering a plurality of viral particles to a mammal as set forth herein, where severity, frequency, progression or time of onset of one or more symptoms of a disease state, such as a neuro-degenerative disease, decreased, reduced, prevented, inhibited or delayed. In certain embodiments, a method includes administering a plurality of viral particles to a mammal to treat an adverse symptom of a disease state, such as a neuro-degenerative disease. In certain embodiments, a method includes administering a plurality of viral particles to a mammal to stabilize, delay or prevent worsening, or progression, or reverse and adverse symptom of a disease state, such as a neuro-degenerative disease.

In certain embodiments a method includes administering a plurality of viral particles to the central nervous system, or portion thereof as set forth herein, of a mammal and severity, frequency, progression or time of onset of one or more symptoms of a disease state, such as a neuro-degenerative disease, are decreased, reduced, prevented, inhibited or delayed by at least about 5 to about 10, about 10 to about 25, about 25 to about 50, or about 50 to about 100 days.

In certain embodiments, a symptom or adverse effect comprises an early stage, middle or late-stage symptom; a behavior, personality or language symptom; swallowing, movement, seizure, tremor or fidgeting symptom; ataxia; and/or a cognitive symptom such as memory, ability to organize.

In certain embodiments, a method includes administering or delivering AAV-TPP1 particles to a mammal and administering one or more immunosuppressive agents to the mammal. In certain embodiments a method includes administering or delivering AAV-TPP1 particles to a mammal and administering 2, 3, 4 or more immunosuppressive agents to the mammal. In certain embodiments a method includes administering or delivering AAV-TPP1 particles to a mammal and administering two immunosuppressive agents to the mammal. In one representative embodiment, a method of treating a mammal includes administering or delivering AAV-TPP1 particles to a mammal and administering first and second immunosuppressive agents to the mammal.

Where two or more immunosuppressive agents are administered, each immunosuppressive agent is distinct and/or different (e.g., each agent differs in structure and/or mechanism of action). An “agent” refers to an active pharmaceutical ingredient. In certain embodiments, an immunosuppressive agent is an anti-inflammatory agent. In certain embodiments, an immunosuppressive agent is mycophenolate, or a derivative thereof. An example of such a mycophenolate derivative is mycophenolate mofetil (MMF). In certain embodiments, an immunosuppressive agent is cyclosporine or a derivative thereof. In certain embodiments a first immunosuppressive agent comprises cyclosporine and a second immunosuppressive agent comprises mycophenolate, or a derivative thereof (e.g., MMF). In certain embodiments a first immunosuppressive agent comprises cyclosporine and a second immunosuppressive agent comprises MMF.

In certain embodiments, an immunosuppressive agent is administered before, during and/or after administration of AAV-TPP1 particles to a mammal. In certain embodiments, an immunosuppressive agent is administered concurrently with administration of AAV-TPP1 particles to a mammal. In certain embodiments, an immunosuppressive agent is administered after administration of AAV-TPP1 particles to a mammal. In certain embodiments, an immunosuppressive agent is administered about 1 to about 60 minutes after, about 1 to about 24 hours after, about 1 to about 100 days after, about 1 to about 12 months after, or about 1 to about 5 years after administration of AAV-TPP1 particles to a mammal. In certain embodiments, an immunosuppressive agent is administered before administration of AAV-TPP1 particles to a mammal. In certain embodiments, an immunosuppressive agent is administered about 1 to about 60 minutes before, about 1 to about 24 hours before, about 1 to about 100 days before, or about 1 to about 3 months before administration of AAV-TPP1 particles to a mammal. In certain embodiments, an immunosuppressive agent is administered about 1, about 2, about 3, about 4, about 5, about 6, about 7, about 8, about 9 or about 10 days before administration of AAV-TPP1 particles to a mammal. In certain embodiments, an immunosuppressive agent is administered at predetermined intervals before, during and/or after administration of AAV-TPP1 particles to a mammal (e.g., once a day, twice a day, three times a day, every other day, weekly, biweekly, bi-monthly, combination thereof or the like).

In certain embodiments a first immunosuppressive agent is administered to a mammal at least about 1 to about 7 days before, or about 1, about 2, about 3, about 4 or about 5 weeks before administration of AAV-TPP1 particles to a mammal and a second immunosuppressive agent is administered about 1 to about 7 days before, about 1, about 2, about 3, about 4 or about 5 weeks before, during and/or within about 10, about 20, about 30, about 40, 19 about 50, about 100, about 200, about 300, about 350, about 400 or about 500 days after administration of AAV-TPP1 particles to the mammal. In certain embodiments cyclosporine is administered to a mammal at least about 1 to about 7 days before, or about 1, about 2, about 3, about 4 or about 5 weeks before administration of AAV-TPP1 particles to a mammal, and mycophenolate or a derivative thereof (e.g., MMF) is administered about 1 to about 7 days before, about 1, about 2, about 3, about 4 or about 5 weeks before, during and/or within about 10, about 20, about 30, about 40, about 50, about 100, about 200, about 300, about 350, about 400 or about 500 days after administration of AAV-TPP1 particles to the mammal. In certain embodiments, cyclosporine is administered about 1 to about 7 days before, or about 1, about 2, about 3, about 4 or about 5 weeks before administration of AAV-TPP1 particles and at regular intervals after treatment, and mycophenolate or a derivative thereof (e.g., MMF) is administered once at about 1 to about 7 days before, about 1, about 2, about 3, about 4 or about 5 weeks before, during and/or within about 10 to about 40 days after administration of AAV-TPP1 particles to the mammal.

An immunosuppressive agent can be administered at any suitable dose. In certain embodiments, cyclosporine is administered at a dosage of about 1 to about 50 mg/kg, about 1 to about 20 mg/kg, or about 5 to about 10 mg/kg at a frequency of once, twice or three times a day, to once every other day. In certain embodiments cyclosporine is administered at about 10 mg/kg twice a day. In certain embodiments, cyclosporine is administered at about 10 mg/kg twice a day for a period of at least about 1, about 2, about 3, about 4 or about 5 months. In certain embodiments, a dosage of cyclosporine is tapered down to a dose of less than about 5 mg/kg, or less than about 2 mg/kg about 1 to about 2 months after the administration of AAV-TPP1 particles to a mammal.

In certain embodiments, mycophenolate or a derivative thereof (e.g., MMF), is administered at a dosage of about 1 to about 100 mg/kg, about 1 to about 50 mg/kg, about 1 to about 25 mg/kg, or about 5 to about 20 mg/kg at a frequency of once, twice or three times a day, to once every other day. In certain embodiments, mycophenolate or a derivative thereof (e.g., MMF) is administered at about 10 to about 20 mg/kg once a day. In certain embodiments, a dosage of mycophenolate or a derivative thereof (e.g., MMF) is reduced down to a dose of less than about 5 mg/kg, or less than about 2 mg/kg about 1 to about 2 months after the administration of AAV-TPP1 particles to a mammal. An immunosuppressive agent can be formulated in any suitable formulation suitable for a particular route of administration. Various pharmaceutically acceptable formulations of immunosuppressive agents are commercially available and readily obtainable by a medical practitioner.

An immunosuppressive agent can be administered by any suitable route. In certain embodiments, an immunosuppressive agent is administered orally. In certain embodiments, mycophenolate or a derivative thereof, such as Mycophenolate Mofetil (MMF), is administered orally. In certain embodiments, cyclosporine is administered orally. An immunosuppressive agent can also be administered parenterally (e.g., intramuscularly, intravenously, subcutaneously), or administered by injection to the brain, spinal cord, or a portion thereof (e.g., injected into the CSF).

In certain embodiments, a method includes administering one or more (e.g., a plurality of) AAV-TPP1 particles to the central nervous system of a mammal (e.g., a mammal having a LSD). In certain embodiments, the central nervous system includes brain, spinal cord and cerebral spinal fluid (CSF). In certain embodiments, a method includes administering one or more AAV-TPP1 particles to the brain or spinal cord or CSF of a mammal. In certain embodiments AAV-TPP1 particles are administered to a portion of brain or spinal cord. In certain embodiments, a composition including AAV-TPP1 particles and an immunosuppressive agent are administered to a mammal's cisterna magna and/or to the mammal's brain ventricle, subarachnoid space, and/or intrathecal space, and/or ependyma. For example, AAV-TPP1 particles can be delivered directly to the cisterna magna, intraventricular space, a brain ventricle, subarachnoid space, intrathecal space or ependyma. In certain embodiments a method includes administering one or more AAV-TPP1 particles to the ependyma of a mammal.

In certain embodiments, AAV-TPP1 particles are administered to one or more cells that contact the CSF in a mammal, for example by contacting cells with AAV-TPP1 particles. Nonlimiting examples of cells that contact the CSF include ependymal cells, pial cells, endothelial cells and/or meningeal cells. In certain embodiments AAV-TPP1 particles are administered to ependymal cells. In certain embodiments AAV-TPP1 particles are delivered to ependymal cells, for example by contacting ependymal cells with AAV-TPP1 particles.

In certain embodiments, AAV-TPP1 particles are delivered locally. “Local delivery” refers to delivery of an active agent directly to a target site within a mammal (e.g., directly to a tissue or fluid). For example, an agent can be locally delivered by direct injection into an organ, tissue or specified anatomical location. In certain embodiments one or more AAV-TPP1 particles are delivered or administered by direct injection to the brain, spinal cord, or a tissue or fluid thereof (e.g., CSF, such as ependymal cells, pial cells, endothelial cells and/or meningeal cells). For example, AAV-TPP1 particles can be directly delivered, by way of direct injection, to the CSF, cisterna magna, intraventricular space, a brain ventricle, subarachnoid space and/or intrathecal space and/or ependyma. In certain embodiments AAV-TPP1 particles are contacted with a tissue, fluid or cell of the brain or spinal cord by direct injection into a tissue or fluid of the brain or spinal cord. In certain embodiments AAV-TPP1 particles are not delivered systemically by, for example, intravenous, subcutaneous, or intramuscular injection, or by intravenous infusion. In certain embodiments AAV-TPP1 particles are delivered to a tissue or fluid of the brain or spinal cord by stereotactic injection.

In certain embodiments, one or more AAV-TPP1 particles are delivered or administered by direct injection of AAV-TPP1 particles to the brain, spinal cord, or a tissue or fluid thereof (e.g., CSF such as ependyma). In a particular aspect, AAV-TPP particles transduce ependymal cells, pial cells, endothelial cells and/or meningeal cells.

As is apparent to those skilled in the art in view of the teachings herein, such as the dose ranges provided herein, an effective amount of AAV-TPP1 particles can be empirically determined. Administration can be effected in one dose, continuously or intermittently throughout the course of treatment. Effective doses of administration can be determined by those of skill in the art and may vary according to the AAV serotype, viral titer and the weight, condition and species of mammal being treated. Single and multiple administrations can be carried out with the dose level, target and timing being selected by the treating physician.

In certain embodiments, a plurality of AAV-TPP1 particles are administered. As used herein, a plurality of AAV particle refers to about 1×10⁵ to about 1×10⁸ particles.

In certain embodiments, AAV-TPP1 particles are administered at a dose of about 1×10⁵ to about 1×10¹⁶ vg/ml in about 1 to about 5 ml; at a dose of about 1 to about 3 ml of 1×10⁷ to about 1×10¹⁴ vg/ml; or at a dose of about 1 to about 2 ml of 1×10⁸ to about 1×10¹³ vg/ml In certain embodiments, AAV-TPP1 particles are administered at a dose of about 1×10⁸ to about 1×10¹⁵ vg/kg body weight of the mammal being treated. For example, AAV-TPP1 particles can be administered at a dose of about 1×10⁸ vg/kg, about 5×10⁸ vg/kg, about 1×10⁹ vg/kg, about 5×10⁹ vg/kg, about 1×10¹⁰ vg/kg, about 5×10¹⁰ vg/kg, about 1×10¹¹ vg/kg, about 5×10¹¹ vg/kg, about 1×10¹² vg/kg, about 5×10¹² vg/kg, about 1×10¹³ vg/kg, about 5×10¹³ vg/kg, about 1×10¹⁴ vg/kg, about 5×10¹⁴ vg/kg, or about 1×10¹⁵ vg/kg body weight of the mammal being treated.

Administration of AAV-TPP1 particles may be in one or more doses. Multiple doses may be administered as is required to maintain adequate enzyme activity, for example.

### V. PHARMACEUTICAL COMPOSITIONS

As used herein the term “pharmaceutically acceptable” and “physiologically acceptable” mean a biologically acceptable composition, formulation, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Such composition, “pharmaceutically acceptable” and “physiologically acceptable” formulations and compositions can be sterile. Such pharmaceutical formulations and compositions may be used, for example in administering a viral particle to a subject.

Such formulations and compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Supplementary active compounds (e.g., preservatives, antibacterial, antiviral and antifungal agents) can also be incorporated into the formulations and compositions.

Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as surfactants, wetting or emulsifying agents, pH buffering substances, and the like, may be present in such vehicles.

Pharmaceutical compositions can be formulated to be compatible with a particular route of administration or delivery, as set forth herein or known to one of skill in the art. Thus, pharmaceutical compositions include carriers, diluents, or excipients suitable for administration or delivery by various routes.

Pharmaceutical forms suitable for injection or infusion of AAV-TPP1 particles can include sterile aqueous solutions or dispersions which are adapted for the extemporaneous preparation of sterile injectable or infusible solutions or dispersions, optionally encapsulated in liposomes. In all cases, the ultimate form should be a sterile fluid and stable under the conditions of manufacture, use and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Isotonic agents, for example, sugars, buffers or salts (e.g., sodium chloride) can be included. Prolonged absorption of injectable compositions can be brought about by the use in the compositions of agents delaying absorption, for example, aluminum monostearate and gelatin.

Solutions or suspensions of AAV-TPP1 particles can optionally include the following components: a sterile diluent such as water for injection, saline solution, such as phosphate buffered saline (PBS), artificial CSF, fixed oils, a polyol (for example, glycerol, propylene glycol, and liquid polyethylene glycol, and the like), glycerin, or other synthetic solvents; antibacterial and antifungal agents such as parabens, chlorobutanol, phenol, ascorbic acid, and the like; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

Pharmaceutical formulations, compositions and delivery systems appropriate for the compositions, methods and uses of the disclosure are known in the art (see, e.g., Remington: The Science and Practice of Pharmacy (2003) 20^{th} ed., Mack Publishing Co., Easton, PA; Remington's Pharmaceutical Sciences (1990) 18^{th} ed., Mack Publishing Co., Easton, PA; The Merck Index (1996)₁₂^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., 2004).

AAV-TPP1 particles and compositions may be formulated in dosage unit form for ease of administration and uniformity of dosage. Dosage unit form as used herein refers to physically discrete units suited as unitary dosages for an individual to be treated; each unit containing a predetermined quantity of active compound calculated to produce the desired therapeutic effect in association with the required pharmaceutical carrier. The dosage unit forms are dependent upon the amount of AAV-TPP1 particles necessary to produce the desired effect(s). The amount necessary can be formulated in a single dose or can be formulated in multiple dosage units. The dose may be adjusted to a suitable AAV-TPP1 particles concentration, optionally combined with an anti-inflammatory agent, and packaged for use.

In one embodiment, pharmaceutical compositions will include sufficient genetic material to provide a therapeutically effective amount, i.e., an amount sufficient to reduce or ameliorate symptoms of a disease state in question or an amount sufficient to confer the desired benefit. Pharmaceutical compositions typically contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Thus, for example, viral particles, and pharmaceutical compositions thereof, can be packaged in single or multiple unit dosage form for ease of administration and uniformity of dosage.

Formulations containing AAV-TPP1 particles will contain an effective amount of the rAAV particles in a vehicle, the effective amount being readily determined by one skilled in the art. The AAV-TPP1 particles may typically range from about 1% to about 95% (w/w) of the composition, or even higher if suitable. The quantity to be administered depends upon factors such as the age, weight and physical condition of the mammal or the human subject considered for treatment. Effective dosages can be established by one of ordinary skill in the art through routine trials establishing dose response curves.

### VI. DEFINITIONS

A “promoter” refers to a nucleotide sequence, usually upstream (5′) of a coding sequence, which directs and/or controls the expression of the coding sequence by providing the recognition for RNA polymerase and other factors required for proper transcription. In some embodiments, the promoter comprises a sequence having at least 50% identity, at least 60% identity, at least 70% identity, at least 75% identity, at least 80% identity, at least 85% identity, at least 90% identity, at least 95% identity, at least 98% identity, or 100% identity to the nucleic acid set forth in SEQ ID NO: 1.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Promoters and/or enhancers may be derived in their entirety from a native gene or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

A “transgene” is used herein to conveniently refer to a nucleic acid sequence/polynucleotide that is intended or has been introduced into a cell or organism. Transgenes include any nucleic acid, such as a gene that encodes an inhibitory RNA polypeptide or protein (e.g., TTP1) and are generally heterologous with respect to naturally occurring genomic sequences.

The term “transduce” refers to introduction of a nucleic acid sequence into a cell or host organism by way of a vector (e.g., a viral particle). Introduction of a transgene into a cell by a viral particle can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded protein expressed. For gene therapy uses and methods, a transduced cell can be in a mammal.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein, e.g., a variant TPP1 protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a TPP1 protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide (e.g., TPP1) retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence). Another example of an amino acid modification is a targeting peptide introduced into a capsid protein of a viral particle. Peptides have been identified that target recombinant viral vectors, to the central nervous system, such as to distinct brain regions.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

### VII. KITS

The disclosure provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, and/or viral particles.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, or an electrical storage media such as RAM and ROM or hybrids of these such as magnetic/optical storage media, FLASH memory, hybrids and memory type cards.

### VIII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Evolved AAV Capsids for Gene Therapy of CLN2 Disease

A novel AAV capsid that preferentially transduces ependymal cells as well as neurons in NHPs was developed (described in PCT/US2020/061464). Subsequently, a gene therapy for CLN2 disease was designed (previously described in prior patent whereas the gene therapy was packaged in AAV2), using the AAV capsid and ubiquitous neuronal promoter sequence (CAG) to drive the expression of tripeptidyl peptidase 1 (TPP1) in neurons throughout the brain. As shown in Appendix 1, secreted hTPP1 from transduced ependyma was widely and stably expressed in the brain, well tolerated and produced a dose-dependent therapeutic benefit in TPP1-deficient mice. Peptide modified-AAV resulted in robust improvements in hTPP1 biodistribution, with improved phenotypes at doses significantly lower (5E10vg) than conventional serotypes (1E11 vg) in mice.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### IX. REFERENCES

The following references, to the extent that they provide exemplary procedural or other details supplementary to those set forth herein, are specifically incorporated herein by reference.
U.S. Pat. No. 8,299,215
U.S. Pat. No. 8,691,948
United States Patent Application Publication No. 2018/0142259
Chen et al., Molecular signatures of disease brain endothelia provide new sites for CNS-directed enzyme therapy. Nat Med, 2009. 15(10): p. 1215-8.
Chen et al., Overcoming Limitations Inherent in Sulfamidase to Improve Mucopolysaccharidosis IIIA Gene Therapy. Mol Ther, 2018. 26(4): p. 1118-1126.
Deverman et al., Cre-dependent selection yields AAV variants for widespread gene transfer to the adult brain. Nat Biotechnol, 2016. 34(2): p. 204-9.
Hartz et al., Isolation of Cerebral Capillaries from Fresh Human Brain Tissue. J Vis Exp, 2018(139).
Hordeaux et al., The Neurotropic Properties of AAV-PHP.B Are Limited to C57BL/6J Mice.

Mol Ther, 2018. 26(3): p. 664-668.
Junaid et al., A Novel Assay for Lysosomal Pepstatin-Insensitive Proteinase and its Application for the Diagnosis of Late-Infantile Neuronal Ceroid Lipofuscinosis. Clin Chim Act, 1999. 281: p. 169-176.
Katz et al., AAV gene transfer delays disease onset in a TPP1-deficient canine model of the late infantile form of Batten disease. Sci Transl Med, 2015. 7(313): p. 313r180.
Keiser et al., Broad distribution of ataxin 1 silencing in rhesus cerebella for spinocerebellar ataxia type 1 therapy. Brain, 2015. 138(Pt 12): p. 3555-66.
Koerber et al., DNA shuffling of adeno-associated virus yields functionally diverse viral progeny. Mol Ther, 2008. 16(10): p. 1703-9.
Matsuzaki et al., Intravenous administration of the adeno-associated virus-PHP.B capsid fails to upregulate transduction efficiency in the marmoset brain. Neurosci Lett, 2018. 665: p.182-188.
McBride et al., Preclinical safety of RNAi-mediated HTT suppression in the rhesus macaque as a potential therapy for Huntington's disease. Mol Ther, 2011. 19(12): p. 2152-62.
Monteys et al., CRISPR/Cas9 Editing of the Mutant Huntingtin Allele In Vitro and In Vivo. Mol Ther, 2017. 25(1): p. 12-23.
Muller et al., Random peptide libraries displayed on adeno-associated virus to select for targeted gene therapy vectors. Nat Biotechnol, 2003. 21(9): p. 1040-6.
Poznansky et al., Drug Delivery Systems. R. L. Juliano, ed., Oxford, N.Y., pp. 253-315, 1980.
Sambrook et al., Molecular Cloning, a Laboratory Manual. Cold Spring Harbor, N.Y. 1989.
Schaffer & Maheshri, Directed evolution of AAV mutants for enhanced gene delivery. Conf Proc IEEE Eng Med Biol Soc, 2004. 5: p. 3520-3.
Zhong et al., Next generation of adeno-associated virus 2 vectors: point mutations in tyrosines lead to high-efficiency transduction at lower doses. Proc Natl Acad Sci USA, 2008. 105(22): p. 7827-32.

1. A method of expressing a tripeptidyl peptidase 1 (TPP1) in a neuron and/or ependyma cells of a subject, comprising administering to the subject a modified adeno-associated virus (AAV) encoding a TPP1 under the control of a promoter operable in said neuron, such as central nervous system neurons, or said ependymal cell, wherein said AAV comprises a modified capsid protein comprising the targeting sequence ERDRTRG (SEQ ID NO: 3), and wherein said modified AAV does not target dorsal root ganglia.

2. The method of claim 1, wherein the promoter may be a constitutive promoter.

3. The method of claim 1, wherein the promoter may be a tissue specific promoter.

4. The method of claim 1, wherein the promoter is a CMV early enhancer/chicken R actin (CAG) promoter.

5. The method of claim 4, wherein the CAG promoter comprises the sequence of SEQ ID NO: 1.

6. The method of claim 1, wherein the modified AAV is derived from AAV1.

7. The method of claim 1, wherein the targeting sequence is inserted near or after position 590 of SEQ ID NO: 2.

8. The method of claim 1, wherein the targeting sequence consists essentially of ERDRTRG (SEQ ID NO: 3).

9. The method of claim 1, wherein the targeting sequence consists of ERDRTRG (SEQ ID NO: 3).

10. The method of claim 1, wherein the TPP1 comprises the sequence of SEQ ID NO: 4.

11. The method of claim 1, wherein the TPP1 consists of the sequence of SEQ ID NO: 4.

12. The method of claim 1, wherein the targeting sequence is flanked by linker sequences, wherein the linker sequences on each side of the targeting sequence are two or three amino acids long.

13. The method of claim 12, wherein the linker sequences are SSA on the N-terminal side of the targeting sequence and AS on the C-terminal side of the targeting sequence.

14. The method of claim 1, wherein the modified capsid protein comprises the sequence of SEQ ID NO: 5.

15. The method of claim 1, wherein the modified capsid protein consists of the sequence of SEQ ID NO: 5.

16. The method of claim 1, wherein administering is direct intracerebroventricular or intracisternal magna or intrathecal delivery.

17. The method of claim 1, wherein the modified AAV administering occurs more than once.

18. The method of claim 17, wherein the modified AAV administering comprises 2, 3, 4, 5, 6, 7, 8, 9, 10 or more administrations.

19. The method of claim 17, wherein the modified AAV administering occurs monthly, every other month, every two months, every three months, every four months, every six months, annually every other year, every three years, every four year or every five years.

20. The method of claim 1, wherein a plurality of viral particles are administered.

21. The method of claim 20, wherein administering comprises dosing at about 1×10⁶ to about 1×10¹⁴ vector genomes per kilogram (vg/kg).

22. The method of claim 21, wherein administering comprises dosing from about 1×10⁷-1×10¹⁴, about 1×10⁸-1×10¹⁴, about 1×10⁹-1×10¹⁴, about 1×10¹⁰-1×10¹⁴, about 1×10¹⁰-1×10¹³, about 1×10¹⁰-1×10¹³, about 1×10¹⁰-1×10¹¹, about 1×10¹¹-1×10¹², or about 1×10¹²-1×10¹³ vector genomes per kilogram (vg/kg) of the patient.

23. The method of claim 1, wherein the TPP1 sequence is operably linked to a poly-adenylation signal.

24. The method of claim 1, wherein said subject suffers from late infantile neuronal ceroid lipofusincosis type 2 (CLN2) disease/Batten disease.

25. The method of claim 1, wherein said subject is less than 4 years old, or is between 4 and 25 years old.

26. A modified adeno-associated virus (AAV) encoding a TPP1 under the control of a promoter operable in a neuron or ependyma cell, wherein said AAV comprises a modified capsid protein comprising the targeting sequence ERDRTRG (SEQ ID NO: 3), and wherein said modified AAV does not target dorsal root ganglia.

27. The modified AAV of claim 26, wherein the promoter may be a constitutive promoter.

28. The modified AAV of claim 26, wherein the promoter may be a tissue specific promoter.

29. The modified AAV of claim 26, wherein the promoter is a CMV early enhancer/chicken β actin (CAG) promoter.

30. The modified AAV of claim 29, wherein the CAG promoter comprises the sequence of SEQ ID NO: 1.

31. The modified AAV of claim 26, wherein the modified AAV is AAV1.

32. The modified AAV of claim 26, wherein the targeting sequence is inserted near or after position 590 of SEQ ID NO: 1.

33. The modified AAV of claim 26, wherein the targeting sequence consists essentially of ERDRTRG (SEQ ID NO: 3).

34. The modified AAV of claim 26, wherein the targeting sequence consists of ERDRTRG (SEQ ID NO: 3).

35. The modified AAV of claim 26, wherein the TPP1 comprises the sequence of SEQ ID NO: 4.

36. The modified AAV of claim 26, wherein the TPP1 consists of the sequence of SEQ ID NO: 4.

37. The modified AAV of claim 26, wherein the targeting sequence is flanked by linker sequences, wherein the linker sequences on each side of the targeting sequence are two or three amino acids long.

38. The modified AAV of claim 37, wherein the linker sequences are SSA on the N-terminal side of the targeting sequence and AS on the C-terminal side of the targeting sequence.

39. The modified AAV of claim 26, wherein the modified capsid protein comprises the sequence of SEQ ID NO: 5.

40. The modified AAV of claim 26, wherein the modified capsid protein consists of the sequence of SEQ ID NO: 5.

41. A pharmaceutical composition comprising the modified AAV of any one of claim 26 and a pharmaceutically acceptable carrier.

42. A kit comprising the modified AAV of claim 26.

43. Use of a modified adeno-associated virus (AAV) encoding a tripeptidyl peptidase 1 (TPP1) under the control of a promoter operable in a neuron, wherein said AAV comprises a modified capsid protein comprising the targeting sequence ERDRTRG (SEQ ID NO: 3), for expressing TPP1 in a neuron or ependymal cell, wherein said modified AAV does not target dorsal root ganglia.

44. Use of a modified adeno-associated virus (AAV) encoding a tripeptidyl peptidase 1 (TPP1) under the control of a promoter operable in a neuron or ependymal cell, wherein said AAV comprises a modified capsid protein comprising the targeting sequence ERDRTRG (SEQ ID NO: 3), for treating late infantile neuronal ceroid lipofusincosis type 2 (CLN2) disease/Batten disease in a subject afflicted therewith, wherein said modified AAV does not target dorsal root ganglia.

45. Use of a modified adeno-associated virus (AAV) encoding a tripeptidyl peptidase 1 (TPP1) under the control of a promoter operable in a neuron or ependymal cell, wherein said AAV comprises a modified capsid protein comprising the targeting sequence ERDRTRG (SEQ ID NO: 3), in the preparation of a medicament for treating late infantile neuronal ceroid lipofusincosis type 2 (CLN2) disease/Batten disease in a subject afflicted therewith, wherein said modified AAV does not target dorsal root ganglia.
